(19) United States
(12) Patent Application Publication
(10) Pub. No.: US 2017/0023572 A1
(43) Pub. Date: Jan. 26, 2017

(54) SELENIUM AND SELENIUM-DEPENDENT MOLECULES PREDICT PRESENCE OF MYCOBACTERIA

(71) Applicant: UNIVERSITY OF CENTRAL FLORIDA RESEARCH FOUNDATION, Orlando, FL (US)

(72) Inventor: Saleh Naser, Orlando, FL (US)

(21) Appl. No.: 15/216,237

(22) Filed: Jul. 21, 2016

Related U.S. Application Data
(60) Provisional application No. 62/195,085, filed on Jul. 21, 2015.

(51) Int. Cl. G01N 33/573; G01N 33/84; C12Q 1/68

(52) U.S. Cl. CPC G01N 33/573; C12Q 1/689; G01N 33/84; G01N 2800/065; G01N 2333/908; C12Q 2600/158

(57) ABSTRACT

Compositions and methods for predicting the presence of a mycobacterial infection in a subject are provided. In some embodiments, the method further comprises assaying the sample to directly detect the presence of the mycobacterial infection if infection is predicted. However, as this is a time-consuming and expensive process, the disclosed methods can be used to predict the presence of the mycobacterium prior to confirmation by direct detection, thereby saving time and money. The disclosed method involves assaying a biological sample from the subject for detection of selenium, wherein the presence of selenium in the sample is an indication of mycobacterium in the sample. Once a mycobacterium is predicated, and optionally confirmed by direct detection, the method can further comprising treating the subject with a therapeutically effective amount of an antibiotic.

### CROSS-REFERENCE TO RELATED APPLICATIONS

This application claims benefit of U.S. Provisional Application No. 62/195,085, filed Jul. 21, 2015, which is hereby incorporated herein by reference in its entirety.

### BACKGROUND

Mycobacterium avium subspecies paratuberculosis (MAP) is a bacteria implicated in the etiology of multiple diseases including Crohn's disease and diabetes mellitus in humans [Hermon-Taylor, J., et al. (2000). Can J Gastroenterol, 14(6):521-542; Sechi, L. A., et al. (2008). Clin Infect Dis, 46(1):148-149]. It is also known to be a causative agent of Johne's disease, a bovine disease similar to Crohn's disease [Naser, S. A., et al. (2004). The Lancet, 364(9439):1039-1044]. It is an obligate intracellular pathogen, living inside the macrophages of the infected host [Xu, S., et al. (1994). J Immunol, 153(6):2568-2578]. MAP increases the suitability of the macrophage as a host and prevents its own destruction by preventing the acidification of the phagosome and by preventing the fusion of the lysosome and the phagosome into the phagolysosomal complex [Crowle, A. J., et al. (1991). Infect Immun, 59(5):1823-1831; Frehel, C., et al. (1986). Infect Immun, 52(1):252-262]. They are also resistant to destruction even in an acidified, mature phagolysosome [Gomes, M. S., et al. (1999). Infect Immun, 67(7):3199-3206]. The primary mechanism for the destruction of M. avium resistant to phagolysosomal degradation is the induction of apoptosis of the infected macrophage through a tumor necrosis factor α (TNF-α) dependent mechanism [Fratazzi, C., et al. (1999). J Leukoc Biol, 66(5):763-764; Fratazzi, C., et al. (1997). J Immunol, 158(9):4320-4327]. There is evidence that Mycobacteria evade this host response by inhibiting apoptosis, and by stimulating necrosis, which allows the bacteria to disseminate [Kabara, E., et al. (2012). Front Microbiol, 3; Behar, S. M., et al. (2010). Nature Reviews Microbiology, 8(9):668-674]. Furthermore, in an active infection the body's ability to clear apoptotic cells may be outpaced. The delay in clearance results in the apoptotic cell bodies losing their membrane integrity and becoming secondary necrotic cells [Elliott, M. R., et al. (2010). J Cell Biol, 189(7):1059-1070]. In the case of the apoptosis of an active macrophage, this includes the leaking of lysosomal content, including reactive oxygen species (ROS), leading to inflammation and oxidative stress.

Mycobacteria are slow growing microorganisms, which can require several months for visible colonies to be observed on sold agar media. Molecular techniques including Polymerase Chain reaction (PCR) techniques require extensive time, cost and labor. There is therefore a need for a predictive test of mycobacteria in samples to provide faster and cost-effective alternatives.

### SUMMARY

Compositions and methods for predicting the presence of a mycobacterial infection in a sample, such as a sample from a subject, are provided. In some embodiments, the method further comprises assaying the sample to directly detect the presence of the mycobacterial infection if infection is predicted. However, as this is a time-consuming and expensive process, the disclosed methods can be used to predict the presence of the mycobacterium prior to confirmation by direct detection, thereby saving time and money.

The disclosed method involves assaying a biological sample from the subject for detection of selenium, wherein the presence of selenium in the sample is an indication of mycobacterium in the sample. In some embodiments, the method involves directly detecting the presence of selenium using separation and elemental detection techniques, e.g., high performance liquid chromatography (HPLC) with inductively coupled plasma mass spectrometry (ICP-MS).

In some embodiments, the method involves detecting the presence of a selenoprotein in the sample. For example, selenoproteins can be detected by immunoassay using antibodies or the like that selectively bind the selenoprotein. However, the selenoprotein can also be detected indirectly by assaying for its enzymatic activity. This generally involves the use of a colorimetric assay of the selenoprotein's enzymatic activity.

The disclosed methods are disclosed for use with any mycobacterium. In some cases, the mycobacterium is a slow growing mycobacterium. In some embodiments, the mycobacterium is selected from the group consisting of a Mycobacterium tuberculosis complex, a Mycobacterium avium complex (MAC), a Mycobacterium gordonae clade, a Mycobacterium kansasii clade, a Mycobacterium nonchromogenicum/terrae clade, a Mycolactone-producing mycobacteria, and a Mycobacterium simiae clade. In some embodiments, the mycobacterium is selected from the group consisting of M. bohemicum, M. botniense, M. branderi, M. celatum, M. chimaera, M. conspicuum, M. cookii, M. doricum, M. farcinogenes, M. haemophilum, M. heckeshornense, M. intracellulare, M. lacus, M. leprae, M. lepraemurium, M. lepromatosis, M. malmoense, M marinum, M. monacense, M. montefiorense, M. murale, M. nebraskense, M. saskatchewanense, M. scrofulaceum, M. shimoidei, M. szulgai, M. tusciae, M. xenopi, and M. yongonense.

In some embodiments, the mycobacterium is MAP, and the selenoprotein is a glutathione peroxidase. In these embodiments, MAP can be predicted based on the detection of an increase in glutathione peroxidase cellular activity in a sample from the subject.

Mycobacterial infections are believed to be involved in the pathogenesis of many diseases, including inflammatory bowel disease (e.g., Crohn's disease and ulcerative colitis), tuberculosis, Type I Diabetes Mellitus, and Multiple Sclerosis. Therefore, in some embodiments, the subject of the disclosed method has or is suspected of having inflammatory bowel disease, tuberculosis, Type I Diabetes Mellitus, or Multiple Sclerosis.

Once a mycobacterium is predicated, and optionally confirmed by direct detection, the method can further comprising treating the subject with a therapeutically effective amount of an antibiotic. In addition, the subject's infection can be monitored after treatment with the antibiotic using the disclosed methods to confirm that the treatment is effective.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts reduced and oxidized states of glutathione.

FIG. 2 shows agarose gels illustrating the presence or absence of MAP-IS900 gene following nPCR. The PCR products following the second round of nPCR were analyzed on 2% agarose gel. M represents molecular weight marker in bp. = represents negative control from second round of amplification. − represents negative control from first round of amplification. TE represents TE buffer negative control. + represents positive control prepared from MAP DNA strain ATCC 43015. 1-100 represents patient samples.

FIG. 3A is a Scatter plot of selenium-dependent GPx activity for MAP negative and MAP positive bovine samples. FIG. 3B is a scatter plot of selenium-dependent GPx activity for MAP negative and MAP positive samples among CD patients and healthy relatives. FIG. 3C is a scatter plot of selenium-dependent GPx activity for Healthy and CD individuals. FIG. 3D is a scatter plot of selenium-dependent GPx activity for MAP negative and MAP positive among CD patients. FIG. 3E is a scatter plot of selenium-dependent GPx activity for MAP negative and MAP positive in randomized field study.

FIG. 4 is a bar graph showing average GPx activity levels in plasma samples from blood samples identified as MAP negative and positive individuals according to according to disease status.

### DETAILED DESCRIPTION

Bacterial infections are a major global healthcare problem, and their detection has to be performed in diverse settings and samples preferably with single-instrument-based diagnostic modalities, using sensitive and robust probes. Mycobacterium avium spp. paratuberculosis (MAP) is found within the white blood cells of infected animals with Johne's disease, a form of animal paratuberculosis, which is associated with chronic enteritis, reminiscent of Crohn's disease in humans. In humans, Crohn's disease is a debilitating chronic inflammatory syndrome of the gastrointestinal track and adjacent lymph nodes. The detection of MAP in tissues from patients with Crohn's disease has been extensively reported, including in human peripheral blood. In those studies, MAP was identified by a culture method followed by PCR identification of a MAP genomic marker. The whole process took several months to complete, due to the slow growing nature of this pathogen. Such a slow detection method not only delays the diagnosis, but also slows any potential therapeutic intervention. Likewise, difficulties in detecting an intracellular pathogen, such as MAP, hamper studies aimed at the investigation of the potential role of MAP in Crohn's disease pathology, as well as the pathogen's impact on the dairy and beef industries. Compositions and methods are therefore disclosed for predicting the presence of a mycobacterial infection in a sample, such as a sample from a subject.

As used herein, a “sample” or “test sample” can include, but is not limited to, biological material obtained from an organism or from components of an organism, food sample, or environmental sample (e.g. water sample or any other sample from an environmental source believed to contain a microorganism). The test sample may be of any biological tissue or fluid, for example. In some embodiments, the test sample can be a sample from a subject. Examples of sample from a subject include, but are not limited to sputum, cerebrospinal fluid, blood, blood fractions such as serum and plasma, blood cells, tissue, biopsy samples, urine, peritoneal fluid, pleural fluid, amniotic fluid, vaginal swab, skin, lymph fluid, synovial fluid, feces, tears, organs, or tumors. A test sample can also include recombinant cells, cell components, cells grown in vitro, and cell culture constituents including, for example, conditioned medium resulting from the growth of cells in cell culture medium.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “predict” does not refer to the ability to predict the presence of a mycobacterial infection with 100% accuracy. Instead, the skilled artisan will understand that the term “predict” refers to an increased probability that a sample has a mycobacterial infection.

The term “infection” refers to a microbial invasion of living tissue that is deleterious to the organism.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

Selenium Measurement

The measurement of Selenium and/or Selenium-dependent glutathione peroxidase/molecules can be performed using standard methods available in the market.

In some embodiments, the method involves directly detecting the presence of selenium using separation and elemental detection techniques. Suitable separation techniques include high performance liquid chromatography (HPLC), gas chromatography (GC), and capillary electrophoresis (CE). Suitable elemental detection techniques include any type of mass spectrometry, including but not limited to matrix assisted laser desorption time of flight (MALDI-TOF) mass spectrometry, electrospray mass spectrometry, inductively coupled plasma mass spectrometry (ICP/MS), ICP-atomic emission spectrometry (ICP/AES), atomic fluorescence spectrometry (AFS), and atomic absorption spectrometry (AAS). For example, HPLC-ICP/MS can be used for the detection and speciation of selenium in the sample.

In some embodiments, the method involves detecting the presence of a selenoprotein in the sample. For example, selenoproteins can be detected by immunoassay using antibodies or the like that selectively bind the selenoprotein.

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbant assays (ELISAs), radioimmunoassays (MA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson -; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyde Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; 565A; 565C; 565L; 565T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; True Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the aspect include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, N G, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods.

Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, P T and D R Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. For descriptions of ELISA procedures, see Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, 1980; Butler, J. E., In: Structure of Antigens, Vol. 1 (Van Regenmortel, M., CRC Press, Boca Raton, 1992, pp. 209-259; Butler, J. E., In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991); Crowther, “ELISA: Theory and Practice,” In: Methods in Molecule Biology, Vol. 42, Humana Press; New Jersey, 1995; U.S. Pat. No. 4,376,110, each of which is incorporated herein by reference in its entirety and specifically for teachings regarding ELISA methods.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunocomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunocomplex (antigen/antibody) formation. Detection of the immunocomplex then requires a labeled secondary binding agent or a secondary binding agent in conjunction with a labeled third binding agent.

“Under conditions effective to allow immunocomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunocomplexes between the test sample and the originally bound material, and subsequent washing, the occurrence of even minute amounts of immunocomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunocomplex with a labeled antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Az.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), Trinectins' based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilized on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

In some embodiments, the selenoprotein can also be detected indirectly by assaying for its enzymatic activity. This generally involves the use of a colorimetric assay using an enzymatic substrate of the selenoprotein. For example, glutathione peroxidase (GPx) can be detected using a colorimetric assay kit, such as the Sigma-Aldrich Glutathione Peroxidase Cellular Activity Assay Kit (Sigma-Aldrich, St. Louis, Mo., USA). In this case, GPx converts reduced glutathione (GSH) to oxidized glutathione (GSSG) while reducing lipid hydroperoxides to their corresponding alcohols or free hydrogen peroxide to water. The generated GSSG is then reduced to GSH with consumption of NADPH by glutathione reductase (GR). The decrease of NADPH (easily measured at 340 nm) is therefore proportional to GPx activity. Colorimetric assays are available or can be developed for other selenoproteins.

The disclosed methods are disclosed for use with any mycobacterium. In some cases, the mycobacterium is a slow growing mycobacterium. In some embodiments, the mycobacterium is selected from the group consisting of a Mycobacterium tuberculosis complex, a Mycobacterium avium complex (MAC), a Mycobacterium gordonae clade, a Mycobacterium kansasii clade, a Mycobacterium nonchromogenicum/terrae clade, a Mycolactone-producing mycobacteria, and a Mycobacterium simiae clade. In some embodiments, the mycobacterium is selected from the group consisting of M. bohemicum, M. botniense, M. branderi, M. celatum, M. chimaera, M. conspicuum, M. cookii, M. doricum, M. farcinogenes, M. haemophilum, M. heckeshornense, M. intracellulare, M. lacus, M. leprae, M. lepraemurium, M. lepromatosis, M. malmoense, M marinum, M. monacense, M. montefiorense, M. murale, M. nebraskense, M. saskatchewanense, M. scrofulaceum, M. shimoidei, M. szulgai, M. tusciae, M. xenopi, and M. yongonense.

The Mycobacterium genus comprises more than 120 different species and is distributed worldwide. Among them are pathogenic species which can cause serious diseases in humans and animals. For example tuberculosis is caused by the Mycobacterium tuberculosis (TB) complex (i.e. M. tuberculosis, M. africanum, M. bovis, M. canettii, M. microti, M. caprae, M. orygis, and M. pinnipedii). The classic Hansen's strain of leprosy is caused by Mycobacterium leprae.

Nontuberculous Mycobacteria (NTM) refers to all the other species in the family of mycobacteria that may cause disease. Every year in the United States approximately two people per 100,000 population develop mycobacterioses caused by these lesser-known “cousins” of TB and leprosy. N™ produces the following major clinical disease syndromes: chronic bronchopulmonary disease, cervical or other lymphadenitis, skin and soft tissue disease, skeletal infection, disseminated infection, and catheter-related infections. Clinical features are dependent on the organism and the site of infection, but are usually chronic and have a progressive clinical course. Being classical opportunists, NTM predominantly infect patients already suffering from pulmonary diseases or immunodeficiency (e.g., HIV-infection) or other chronic antecedent illness. The number of mycobacterioses is increasing among immunocompetent person. Furthermore, NTM infections are emerging in previously unrecognized settings, with new clinical manifestations.

Most infections appear to be acquired by ingestion, aspiration, or inoculation of the organisms from these natural sources; however the specific source of individual infections is usually not identified. No evidence of person-to-person transmission has been reported. Tap water is considered the major reservoir for the most common human NTMs. Species from tap water include M. gordonae, M. kansasii, M. xenopi, M. simiae, M. avium complex, and rapidly-growing Mycobacterium, especially M. mucogenicum. M kansasii, M. xenopi, and M. simiae are recovered almost exclusively from municipal water source

Mycobacterium avium subspecies paratuberculosis (MAP) causes a chronic disease of the intestines in dairy cows and a wide range of other animals, including nonhuman primates, called Johne's (“Yo-knee's”) disease. At least 35% of cattle in USA are infected with MAP. MAP has also been consistently identified in humans with Crohn's disease. The research investigating the presence of MAP in patients with Crohn's disease has often identified MAP in the “negative” ulcerative colitis controls as well, suggesting that ulcerative colitis is also caused by MAP.

Recent findings have also suggested that MAP infection could act as risk factor in favoring multiple sclerosis (MS) progression.

The disclosed method can be used to predict the presence of a mycobacterium to diagnose a disease caused by a mycobacterium. In cases where a disease or disorder is a risk factor for mycobacterial infection, the disclosed methods can be used to make this determination. In some embodiments, the disclosed method can be used to distinguish a mycobacterial related bowel condition from a non-mycobacterial related bowel condition in a patient exhibiting symptoms of a bowel condition. In a specific example, the mycobacterial related bowel condition is inflammatory bowel disease (IBD). In an even more specific example, the bowel condition is Crohn's disease or ulcerative colitis. A patient exhibiting symptoms of a bowel condition typically will exhibit one or more of the following symptoms: abdominal pain, vomiting, diarrhea, rectal bleeding, severe internal cramps/muscle spasms in the region of the pelvis, weight loss and various associated complaints or diseases like arthritis, pyoderma gangrenosum, porridge-like stool, and primary sclerosing cholangitis.

In some embodiments, the method further comprises assaying the sample to directly detect and confirm the presence of the mycobacterial infection if infection is predicted. For example, mycobacterial infection can be detected by culturing the sample in a mycobacterial culture medium (e.g., BACTEC 13A media), and then measuring a time to growth detection.

In some cases, the mycobacterium is detected by a polymerase chain reaction (PCR) method. For example, PCR methods and primers for detecting the presence of Mycobacterium avium subspecies paratuberculosis (MAP) in a sample is described in U.S. Pat. No. 7,488,580 to Naser, which is incorporated by reference in its entirety for the teaching of these methods and primers.

Compositions and method for detecting microbacterial organisms, including MAP, using magnetic relaxation nanosensor (hMRS) adapted to detect a target nucleic acid analyte, are disclosed in U.S. 2014/0220565 by Naser et al., which is incorporated by reference in its entirety for the teaching of these methods and nanosensors.

Upon determining that the bowel condition is a mycobacterial related bowel condition, a therapeutically effective amount of an antibiotic composition can be administered to the patient. Mycobacterial infections are notoriously difficult to treat. The organisms are hardy due to their cell wall, which is neither truly Gram negative nor positive. In addition, they are naturally resistant to a number of antibiotics that disrupt cell-wall biosynthesis, such aspenicillin. Due to their unique cell wall, they can survive long exposure to acids, alkalis, detergents, oxidative bursts, lysis by complement, and many antibiotics. Examples of antibiotics that can in some embodiments be used to treat a mycobacterial infection, include, but are not limited to, metronidazole, ciprofloxacin, rifaximin, rifabutin, clarithromycin, and metronidazole/ciprofloxacin combination, vancomycin, azathioprine, infliximab, tobramycin, or combinations thereof. Most mycobacteria are susceptible to the antibiotics clarithromycin and rifamycin, but antibiotic-resistant strains have emerged.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Oxidative Stress Due to Mycobacterium avium Subspecies Paratuberculosis (MAP) Infection Upregulates Selenium-Dependent GPx Activity

Methods

Bovine Samples

Sera samples from healthy and MAP infected cattle were obtained. Bovine samples were confirmed for MAP infection using the IDEXX Mycobacterium paratuberculosis (M. pt.) Antibody Test Kit (IDEXX Laboratories, Westbrook, Me., USA) following manufacturer instructions. A S/P less than or equal to 0.60 was considered negative and a S/P greater than or equal to 0.70 was considered positive. Sera from 21 MAP infected cattle and 21 healthy cattle were then included in this study.

Human Samples

Human blood samples were collected in two separate sets where each subject provided three 6.0-ml K2-EDTA tubes. All clinical samples were collected following University of Central of Florida-Institutional Review Board approval number IRB00001138. A total of 27 human blood samples were collected from CD patients along with 27 samples of their healthy biological family members (parents or siblings), those samples were collected at the University of Florida (UF). An additional randomized 100 blood samples used in earlier studies were also included. Clinical samples were collected blindly with no prior knowledge of MAP diagnosis or other health conditions. Buffy coat preparations and plasma samples were separated and stored at −20° C.

DNA extraction for PCR analysis was performed on purified buffy coat samples. Each sample was re-suspended in 100 μL of TE buffer and then incubated at 100° C. for 30 min. The re-suspended solution was then placed in an ice bath for 15 min, after which it was centrifuged for 10 min at 4° C. at 12,000 rpm (18,500 g). After centrifugation, the supernatant was extracted in 200 μL of phenol/chloroform/isoamyl alcohol (1:1:24 v/v; Acros Organics, Morris Plains, N.J., USA) was added. The solution was mixed and centrifuged for 5 min at 4° C. at 12,000 rpm (18,500 g). The pellet, containing the nucleic acid, was then washed, dried, and re-suspended in 50 μL of sterile water [Cossu A, et al. Clin Immunol. 2011 141(1):49-57].

Detection of MAP DNA using nested PCR (nPCR) was based on the MAP-specific IS900 derived oligonucleotide primers [Cossu A, et al. Clin Immunol. 2011 141(1):49-57]. As shown in Table 1, P90 and P91 primers were used for the amplification of 398 bp in the first used to amplify a 298 bp internal sequence. Each primary PCR reaction used 10 μL of DNA template and 40 μL of PCR buffer, which consists of 5 mM MgCl2, 0.2 mM dNTP, 2 μM primers, and 2.5 U Platinum Taq polymerase (Invitrogen, Carlsbad, Calif., USA) or 1 U TFL DNA polymerase (Promega, Madison, Wis., USA). Each secondary round of PCR used the same ingredients, except different primers were used and 5 μL of the product of the primary round was used instead of the DNA template. Negative controls for the PCR were prepared in which sterile water or TE buffer was added instead of the DNA template (in the primary amplification) or the primary product (in the secondary amplification). These negatives were prepared in parallel with the samples. Positive controls were also prepared using MAP DNA from strain ATCC 43015. The amplification product size was assessed on 2% agarose gel.

TABLE 1
Primers and amplification conditions used for PCR

| | | | Product |
|---|---|---|---|
| Primer | Oligonucleotide sequence (5′-3′) | Amplification conditions | size (bp) |
| P90, | GTTCGGGGCCGTCGCTTAGG | 95° C. for 5 min, then 34 | 398 |
| P91 | (SEQ ID NO: 1), | cycles of 95° C. for 1 min, | |
| | GAGGTCGATCGCCCACGTGA | 58° C. for 1.5 min, 72° C. | |
| | (SEQ ID NO: 2) | for 1.5 min. Final extension | |
| | | of 10 min at 72° C. | |
| AV1, | ATGTGGTTGCTGTGTTGGATGG | 95° C. for 5 min, then 34 | 298 |
| AV2 | (SEQ ID NO: 3), | cycles of 95° C. for 1 min, | |
| | CCGCCGCAATCAACTCCAG | 58° C. for 1.5 min, 72° C. | |
| | (SEQ ID NO: 4) | for 1.5 min. Final extension | |
| | | of 10 min at 72° C. | |

Selenium-Dependent GPx Activity Measurement

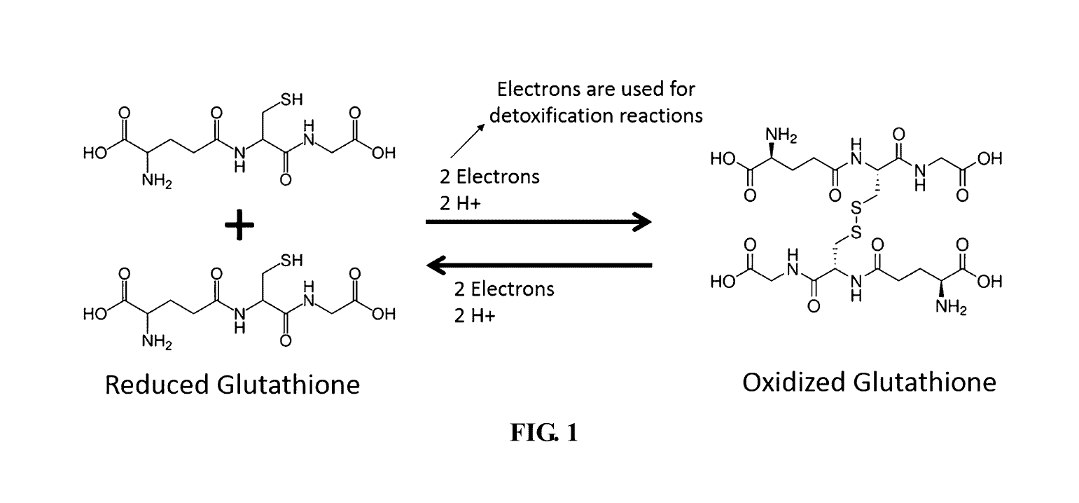

Glutathione peroxidase works by reducing peroxides by oxidizing glutathione. The glutathione is then restored for further cycles of catalysis (FIG. 1). The rate-limiting step of this reaction is that in which the oxidized glutathione used to reduce the peroxide is restored via the oxidation of NADPH. NADPH absorbs at 340 nm. The selenium-dependent GPx activity was measured by using the Sigma-Aldrich GPx Cellular Activity Assay Kit (Sigma-Aldrich, St. Louis, Mo., USA) following manufacturer instructions.

Statistical Analysis

Samples were analyzed for significance using unpaired, two-tailed t tests. SigmaPlot software was used. P values of less than 0.05 were considered significant.

Results

MAP Prevalence in Human Samples

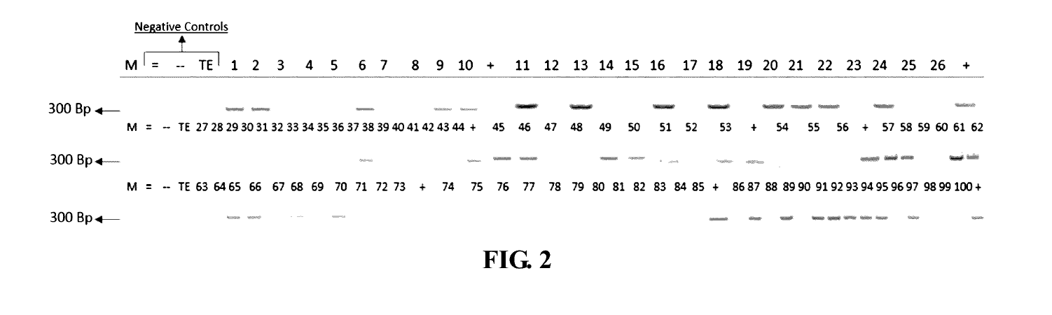

nPCR was performed on DNA extracts isolated from all human blood samples in order to analyze for the presence of MAP-specific IS900 gene according to Naser et al. protocol [Naser S A, et al. Gut Pathog. 2013 5(1):14]. The overall prevalence of MAP among 154 human blood samples was 32%. MAP was positive in the blood of 40% of CD patients compared to 29.9% in non-CD patients. Specifically MAP was also positive in 11/27 (40%) of CD patients and in 2/27 (7%) in healthy biological family members. Interestingly, 33% (7 out of 21) of patients with type II diabetes and 44% (7 out of 16) pre-diabetic patients were also MAP positive. Patients were considered to be pre-diabetic if they had a fasting blood sugar level between 100 and 125 mg/dl, if the two-hour glucose levels was between 140 and 199 mg/dl in an oral glucose tolerance test, or if they had a glycated hemoglobin (A1C) level between 5.7 and 6.4. FIG. 2 illustrates the detection of MAP IS900 gene on 2% agarose gel following nPCR analysis of 100 randomized human blood samples (lanes 1-100).

Selenium-Dependent GPx Levels were Elevated in MAP Infected Bovine Samples

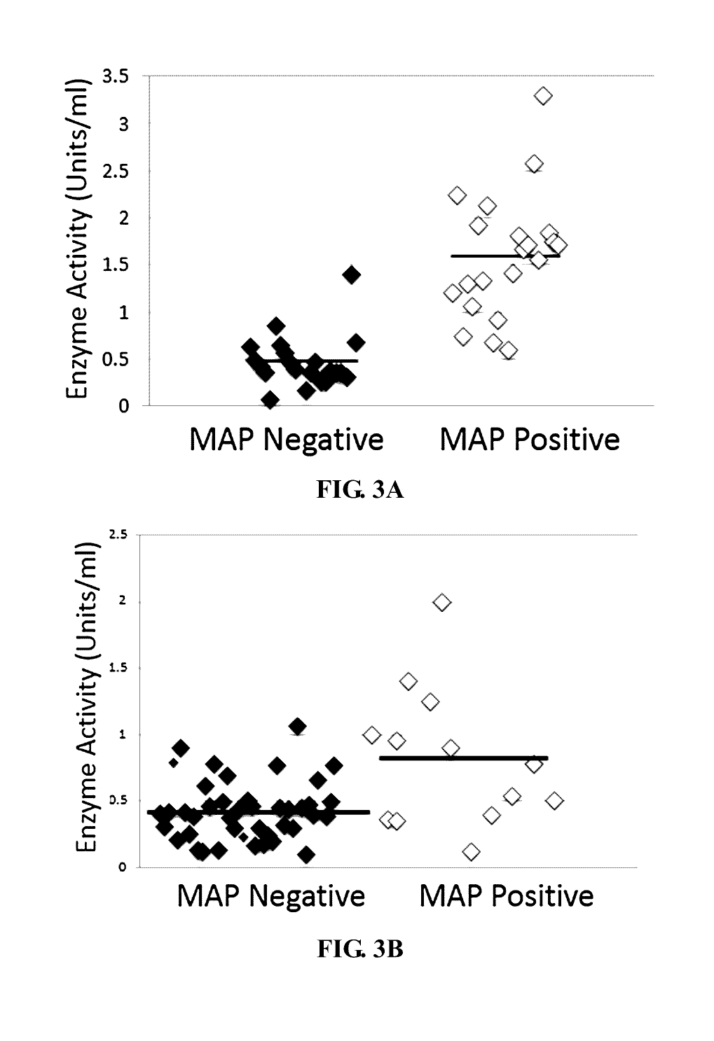

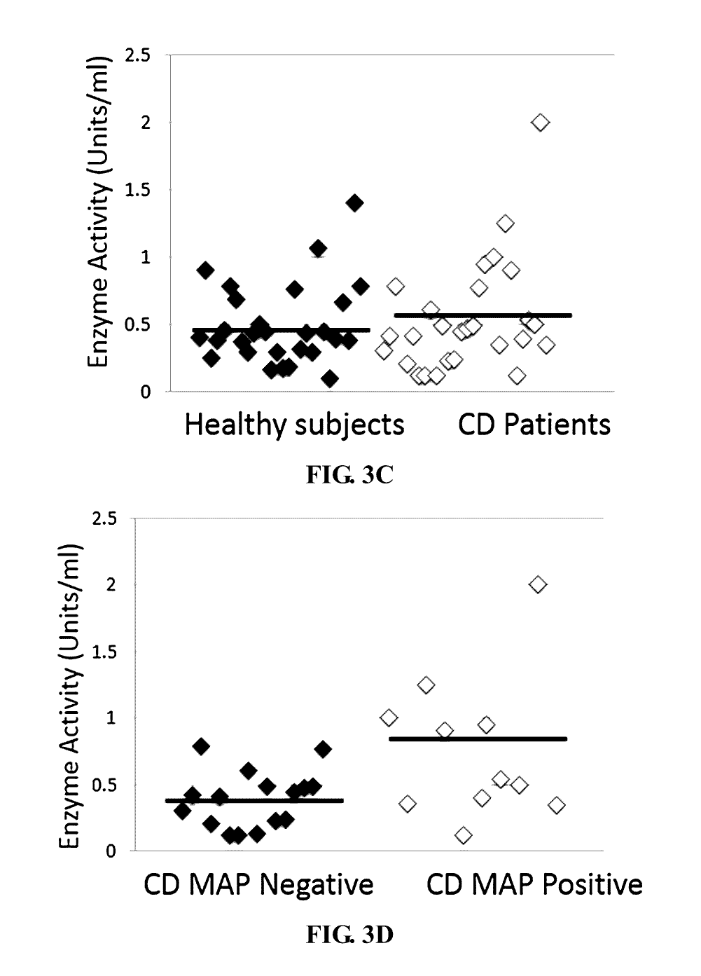

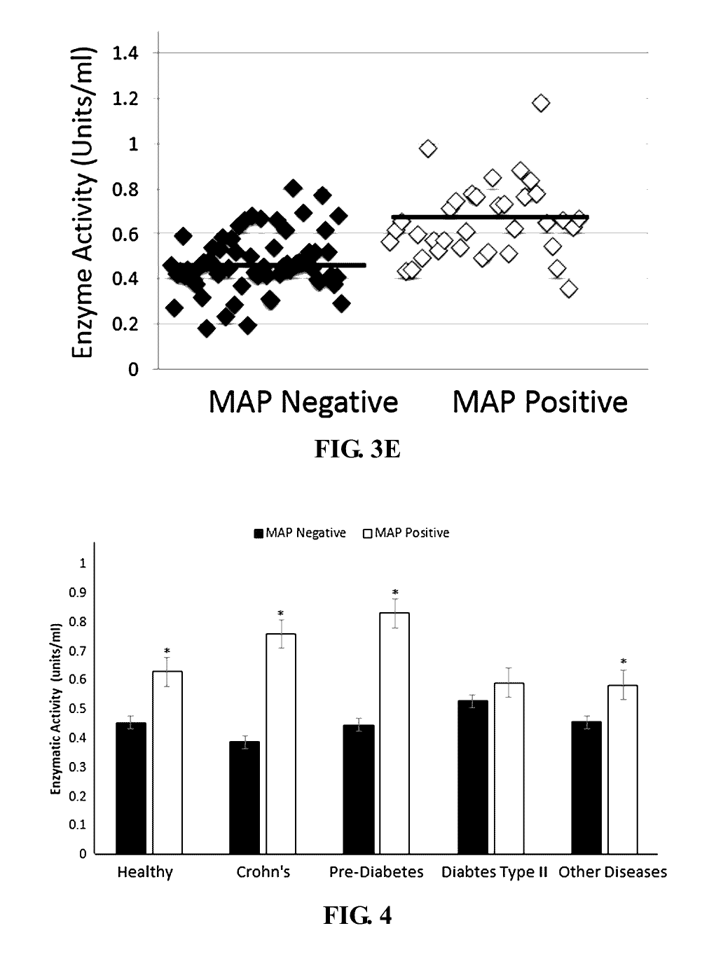

Bovine sera were confirmed for presence of anti-MAP IgG. Consequently, a total of 21 cattle sera samples from animals diagnosed with Johne's disease (MAP positive) and 21 sera from healthy cattle (MAP negative) were selected for the study. All 42 sera were analyzed for of GPx activity. The average level of GPx was 0.46907±0.28 units/ml in healthy cattle sera control compared to 1.590±0.65 units/ml in sera from cows infected with MAP, where a unit was defined as one mmol/minute. The MAP positive samples had a significantly higher activity level, with a difference in means of 1.122 (95% confidence interval 0.810-1.435; P<0.01) (Table 2). FIG. 3a shows a scatter plot of selenium-dependent GPx activity for MAP negative and MAP positive samples.

TABLE 2
GPx enzyme average activity and MAP presence in bovine
and human blood samples

| | | | | Average |
|---|---|---|---|---|
| Number of | | MAP | Average GPx | GPx activity |
| samples/total | Source | diagnosis | activity (units/ml) | (units/ml) P value |
| 21/42 | Bovine | Negative | 0.469 ± 0.28 | <0.01 |
| 21/42 | Bovine | Positive | 1.590 ± 0.65 | |
| 105/154 | Human | Negative | 0.452 ± 0.176 | <0.01 |
| 49/154 | Human | Positive | 0.693 ± 0.30 | |
| 16/27 | CD | Negative | 0.389 ± 0.213 | <0.05 |
| | patients | | | |
| 11/27 | CD | Positive | 0.7593 ± 0.537 | |
| | patients | | | |

Selenium-Dependent GPx Activity was Elevated in MAP Infected Humans Among Crohn's Patients and their Healthy Relatives

The average level of GPx activity was 0.80941±0.521 units/ml in the MAP positive samples, while the average enzyme activity in MAP negative samples was found to be 0.42367±0.229 units/ml. This result reveals that MAP infection has a significant influence on GPx activity, with a difference in means of 0.387 (95% confidence interval 0.182-0.592; P<0.01) (FIG. 3b).

The Difference Between Selenium-Dependent GPx Activity in Crohn's Disease and in Healthy Individuals was not Significant

In order to confirm that the elevation of GPx activity level was due to MAP infection alone, and not due to CD status, the average of GPx activity was measured in healthy individuals and CD patients separately. The average GPx activity was found to be 0.54±0.414 units/ml and 0.493±0.301 units/ml in CD and healthy patients respectively. While the mean GPx enzymatic activity in CD patients was higher by 0.0469, results showed that there was no significant difference between both groups (95% confidence interval −0.245 to 0.151; P=0.636) (FIG. 3c). The gender ratio and age distribution between the two groups was comparable between the two groups (Table 3).

TABLE 3
Demographics of Crohn's patients and healthy relatives

| Group | Age range | Average age | Gender ratio (M/F) |
|---|---|---|---|
| Relatives | 12-65 | 45 | 9/18 |
| Crohn's | 16-56 | 32 | 8/19 |

Selenium-Dependent GPx Activity was Elevated in MAP Infected Crohn's Patients

As mentioned earlier, out of 27 CD patients, a total of 11 were tested as MAP positive, while 16 were MAP negative. The average GPx activity in CD patients who had the MAP infection was 0.7593±0.537 units/ml, while the GPx activity was found to be 0.389±0.213 units/ml in CD patients without MAP infection. The difference in means was 0.37 (95% confidence interval 0.07-0.675; P=0.019). (P=0.019) (FIG. 3d). Furthermore only 2 of the 27 healthy relatives used as controls, or 7.4%, were infected with MAP.

Selenium-Dependent GPx Activity was Elevated Among MAP Infected Humans in Randomized Field Study

Among randomized blood samples from 100 subjects, 36 were determined to be MAP positive as shown in FIG. 2. The average of GPx activity level in 36 MAP positive clinical samples was 0.6510±00.1665 units/ml compared 0.4702±0.1299 in 64 MAP negative clinical samples (P<0.01) (Table 2). The GPx activity in each clinical sample is illustrated in FIG. 3e. The difference in GPx activity was further examined according to disease diagnosis, but there was no significant difference in MAP negative clinical samples between healthy controls and subjects with diseases. Disease states, including type 2 diabetes and pre-diabetes, were not found to have a significant impact on GPx activity. It is notable, however, that in all disease states MAP positive individuals still have higher enzymatic activity than MAP negative individuals (FIG. 4).

### CONCLUSION

The GPx enzymatic activity of selenium dependent GPx was significantly higher in both bovine and human serum samples infected with MAP. The consistent correlation between MAP infection and GPx activity may be used to predict MAP infection status. The presence of this bacterium causes systemic inflammation and oxidative stress, which on the long-term may cause disruptions in insulin signaling and have a deleterious effect on insulin sensitivity. Via this process MAP infection could be involved in the pathophysiology of insulin resistance and in the elevation of oxidative stress level in CD patients who are infected with MAP.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating a mycobacterial infection in a subject, comprising
(a) assaying a biological sample from the subject for the presence of selenium, wherein the presence of selenium in the sample is an indication of mycobacterium in the sample, and
(b) administering to the subject with an effective amount of an antibiotic to treat a mycobacterial infection if selenium is detected in the sample.

2. The method of claim 1, wherein the assay comprises detecting the presence of selenium by high-performance liquid chromatography (HPLC).

3. The method of claim 1, wherein the assay comprises detecting the presence of a selenoprotein in the sample.

4. The method of claim 3, wherein the selenoprotein comprises a glutathione peroxidase.

5. The method of claim 3, wherein the selenoprotein is indirectly detected by a colorimetric assay of the selenoprotein's enzymatic activity.

6. The method of claim 3, wherein the selenoprotein is detected by an immunoassay comprising an antibody that selectively binds the selenoprotein.

7. The method of claim 1, wherein the biological sample is a bodily fluid or tissue sample.

8. The method of claim 1, wherein the mycobacterium is a slow growing mycobacterium.

9. The method of claim 1, wherein the mycobacterium is selected from the group consisting of a Mycobacterium tuberculosis complex, a Mycobacterium avium complex (MAC), a Mycobacterium gordonae clade, a Mycobacterium kansasii clade, a Mycobacterium nonchromogenicum/terrae clade, a Mycolactone-producing mycobacteria, and a Mycobacterium simiae clade.

10. The method of claim 1, wherein the mycobacterium is selected from the group consisting of M. bohemicum, M. botniense, M. branderi, M. celatum, M. chimaera, M. conspicuum, M. cookii, M. doricum, M. farcinogenes, M. haemophilum, M. heckeshornense, M. intracellulare, M. lacus, M. leprae, M. lepraemurium, M. lepromatosis, M. malmoense, M. marinum, M. monacense, M. montefiorense, M. murale, M. nebraskense, M. saskatchewanense, M. scrofulaceum, M. shimoidei, M. szulgai, M. tusciae, M. xenopi, and M. yongonense.

11. The method of claim 1, wherein the subject has or is suspected of having inflammatory bowel disease, tuberculosis, Type I Diabetes Mellitus, or Multiple Sclerosis.

12. The method of claim 1, wherein the biological sample comprises a blood, serum, or plasma sample.

13. A method for treating a mycobacterial infection in a subject, comprising selecting a subject identified as having detectable levels of selenium in their blood, serum, or plasma, and administering to the subject an effective amount of an antibiotic to treat a mycobacterial infection.

14. The method of claim 13, wherein the assay comprises detecting the presence of selenium by high-performance liquid chromatography (HPLC).

15. The method of claim 13, wherein the assay comprises detecting the presence of a selenoprotein in the sample.

16. The method of claim 15, wherein the selenoprotein comprises a glutathione peroxidase.

17. The method of claim 15, wherein the selenoprotein is indirectly detected by a colorimetric assay of the selenoprotein's enzymatic activity.

18. The method of claim 15, wherein the selenoprotein is detected by an immunoassay comprising an antibody that selectively binds the selenoprotein.

19. The method of claim 13, wherein the biological sample is a bodily fluid or tissue sample.

20. The method of claim 13, wherein the mycobacterium is a slow growing mycobacterium.

21. The method of claim 13, wherein the mycobacterium is selected from the group consisting of a Mycobacterium tuberculosis complex, a Mycobacterium avium complex (MAC), a Mycobacterium gordonae clade, a Mycobacterium kansasii clade, a Mycobacterium nonchromogenicum/terrae clade, a Mycolactone-producing mycobacteria, and a Mycobacterium simiae clade.

22. The method of claim 13, wherein the mycobacterium is selected from the group consisting of M. bohemicum, M. botniense, M. branderi, M. celatum, M. chimaera, M. conspicuum, M. cookii, M. doricum, M. farcinogenes, M. haemophilum, M. heckeshornense, M. intracellulare, M. lacus, M. leprae, M. lepraemurium, M. lepromatosis, M. malmoense, M. marinum, M. monacense, M. montefiorense, M. murale, M. nebraskense, M. saskatchewanense, M. scrofulaceum, M. shimoidei, M. szulgai, M. tusciae, M. xenopi, and M. yongonense.

23. The method of claim 13, wherein the subject has or is suspected of having inflammatory bowel disease, tuberculosis, Type I Diabetes Mellitus, or Multiple Sclerosis.

24. A method for diagnosing a mycobacterial infection in a subject, comprising assaying a biological sample from the subject for detection of selenium, wherein the presence of selenium in the sample is an indication of mycobacterium in the sample.

25. The method of claim 24, wherein the assay comprises detecting the presence of selenium by high-performance liquid chromatography (HPLC).

26. The method of claim 24, wherein the assay comprises detecting the presence of a selenoprotein in the sample.

27. The method of claim 26, wherein the selenoprotein comprises a glutathione peroxidase.

28. The method of claim 26, wherein the selenoprotein is indirectly detected by a colorimetric assay of the selenoprotein's enzymatic activity.

29. The method of claim 26, wherein the selenoprotein is detected by an immunoassay comprising an antibody that selectively binds the selenoprotein.

30. The method of claim 24, wherein the biological sample is a bodily fluid or tissue sample.

31. The method of claim 24, wherein the mycobacterium is a slow growing mycobacterium.

32. The method of claim 24, wherein the mycobacterium is selected from the group consisting of a Mycobacterium tuberculosis complex, a Mycobacterium avium complex (MAC), a Mycobacterium gordonae clade, a Mycobacterium kansasii clade, a Mycobacterium nonchromogenicum/terrae clade, a Mycolactone-producing mycobacteria, and a Mycobacterium simiae clade.

33. The method of claim 24, wherein the mycobacterium is selected from the group consisting of M. bohemicum, M. botniense, M. branderi, M. celatum, M. chimaera, M. conspicuum, M. cookii, M. doricum, M. farcinogenes, M. haemophilum, M. heckeshornense, M. intracellulare, M. lacus, M. leprae, M. lepraemurium, M. lepromatosis, M. malmoense, M. marinum, M. monacense, M. montefiorense, M. murale, M. nebraskense, M. saskatchewanense, M. scrofulaceum, M. shimoidei, M. szulgai, M. tusciae, M. xenopi, and M. yongonense.

34. The method of claim 24, wherein the subject has or is suspected of having inflammatory bowel disease, tuberculosis, Type I Diabetes Mellitus, or Multiple Sclerosis.

35. The method of claim 24, wherein the biological sample comprises a blood, serum, or plasma sample.


SEQUENCE LISTING (4 sequences)

SEQ ID NO: 1 - DNA, 20 bp, Artificial Sequence
  Synthetic Construct
gttcggggccgtcgcttagg

SEQ ID NO: 2 - DNA, 20 bp, Artificial Sequence
  Synthetic Construct
gaggtcgatcgcccacgtga

SEQ ID NO: 3 - DNA, 22 bp, Artificial Sequence
  Synthetic Construct
atgtggttgctgtgttggatgg

SEQ ID NO: 4 - DNA, 19 bp, Artificial Sequence
  Synthetic Construct
ccgccgcaatcaactccag